(19) United States
(12) Patent Application Publication
(10) Pub. No.: US 2010/0178680 A1
(43) Pub. Date: Jul. 15, 2010

(54) ROTATABLE PERFUSED TIME VARYING ELECTROMAGNETIC FORCE BIOREACTOR AND METHOD OF USING THE SAME

(75) Inventors: Thomas J. Goodwin, Kemah, TX (US); Clayton R. Parker, Safety Harbor, FL (US)

Correspondence Address: LADAS & PARRY LLP, 224 SOUTH MICHIGAN AVENUE, SUITE 1600, CHICAGO, IL, 60604, US

(73) Assignees: REGENETECH, INC., Texas, TX (US); NASA, Houston, TX (US)

(21) Appl. No.: 11/993,906

(22) PCT Filed: Jun. 26, 2006
(86) PCT No.: PCT/US06/24806

§ 371 Date: Jan. 22, 2010

Related U.S. Application Data
(63) Continuation-in-part of application No. 11/169,385, filed on Jun. 29, 2005, now Pat. No. 7,456,019.
(60) Provisional application No. 60/584,572, filed on Jun. 30, 2004.

(51) Int. Cl. C12N 13/00; C12M 1/00

(52) U.S. Cl. 435/173.8; 435/289.1

(57) ABSTRACT

A rotatable perfused time varying electromagnetic force bioreactor with a rotatable perfusable culture chamber and a time varying electromagnetic force source operatively connected to the rotatable perfusable culture chamber. In use, the rotatable perfused time varying electromagnetic force bioreactor supplies a time varying electromagnetic force to the rotatable perfusable culture chamber of the rotatable perfused time varying electromagnetic force bioreactor to expand cells contained therein.

### FIELD OF THE INVENTION

The present invention generally relates to a rotatable perfused time varying electromagnetic force bioreactor and a method for using the same, and more particularly to a method of expanding cells in a rotatable perfused time varying electromagnetic force bioreactor while at the same time subjecting them to a time varying electromagnetic force.

### BACKGROUND OF THE INVENTION

Cell culture processes have been developed for the growth of single cell bacteria, yeast and molds which are resistant to environmental stresses or are encased with a tough cell wall. Mammalian cell culture, however, is much more complex because mammalian cells are more delicate and have more complex nutrient and other environmental requirements in order to maintain viability and cell growth. Large-scale cultures of bacterial type cells are highly developed and such culture processes are less demanding and are not as difficult to cultivate as mammalian cells. Bacterial cells can be grown in large volumes of liquid medium and can be vigorously agitated without any significant damage. Mammalian cells, on the other hand, cannot withstand excessive turbulent action without damage to the cells and are typically provided with a complex nutrient medium to support growth.

In addition, mammalian cells have other special requirements; in particular most animal cells typically prefer to attach themselves to some substrate surface to remain viable and to duplicate. On a small scale, mammalian cells have been grown in containers with small microwells to provide surface anchors for the cells. However, cell culture processes for mammalian cells in such microwell containers generally do not provide sufficient surface area to grow mammalian cells on a sufficiently large scale basis for many commercial or research applications. To provide greater surface areas, microcarrier beads have been developed for providing increased surface areas for the cultured cells to attach. Microcarrier beads with attached cultured cells require agitation in a conventional bioreactor chamber to provide suspension of the cells, distribution of fresh nutrients, and removal of metabolic waste products. To obtain agitation, such bioreactor chambers have used internal propellers or movable mechanical agitation devices which are motor driven so that the moving parts within a chamber cause agitation in the fluid medium for the suspension of the microcarrier beads and attached cells. Agitation the fluid medium may also agitate mammalian cells therein, however, subjecting them to high degrees of fluid shear stress that can damage the cells and limit ordered assembly of these cells according to cell derived energy. These fluid shear stresses arise, for instance, when the fluid media has significant relative motion with respect to chamber walls, internal propellers or movable mechanical agitation devices, or other chamber components. Cells may also be damaged in bioreactor chambers with internal moving parts if the cells or beads with cells attached collide with one another or chamber components.

In addition to the drawbacks of cell damage, bioreactors and other methods of culturing mammalian cells are also very limited in their ability to provide conditions that allow cells to assemble into tissues that simulate the spatial three-dimensional form of actual tissues in an intact organism and at the same time allow cells to multiply at a rate of at least seven times within seven days. Conventional tissue culture processes limit, for similar reasons, the capacity for cultured tissues to, for instance, develop a highly functionally specialized or differentiated state considered crucial for mammalian cell differentiation and secretion of specialized biologically active molecules of research and pharmaceutical interest. Unlike microorganisms, the cells of higher organisms such as mammals form themselves into high order multicellular tissues. Although the exact mechanisms of this self-assembly are not known, in the cases that have been studied thus far, development of cells into tissues has been found to be dependent on orientation of the cells with respect to each other (the same or different type of cell) or other anchorage substrate and/or the presence or absence of certain substances (factors) such as hormones. In summary, no conventional culture process is capable of simultaneously achieving sufficiently low fluid shear stress, sufficient three-dimensional spatial freedom, and for sufficiently long periods for critical cell interactions (with each other or substrates) to allow excellent modeling of in vivo cell and tissue structure, and at the same time, provides accelerated expansion, growth in the size of tissue and/or tissue constructs and/or growth in the number of cells, while maintaining the cell or tissue three dimensional geometry, and cell-to-cell geometry and support.

For example, U.S. Pat. No. 5,155,035, Wolf et al., provides a method for culturing tissues, tissue constructs, and cells utilizing a perfused bioreactor that overcomes prior problems without subjecting the tissue and/or cells to destructive amounts of shear. The Wolf et al. disclosure, however, provides for a very low rate of production. In fact, the Wolf et al. device, and method of using the same, provides an insufficiently low production rate such that it is not of substantial commercial value.

It is highly desirable, therefore, to have a rotatable perfused time varying electromagnetic force bioreactor that has a rotatable perfusable culture chamber and a time varying electromagnetic force (“TVEMF”)-source operatively connected to the rotatable perfusable culture chamber. It is also highly desirable to have a method for expanding cells using a rotatable perfused TVEMF-bioreactor that, when in use, not only achieves sufficiently low fluid shear stress, sufficient three-dimensional spatial freedom, and for sufficiently long periods for critical cell interactions (with each other or substrates), but at the same time, provides accelerated expansion while maintaining the cell or tissue three dimensional geometry, and cell-to-cell geometry and support.

### SUMMARY OF THE INVENTION

The present invention relates to a rotatable perfused TVEMF-bioreactor comprising a rotatable perfusable culture chamber and a TVEMF source operatively connected to the rotatable perfusable culture chamber.

The present invention also relates to a method for expanding cells in a rotatable perfused TVEMF-bioreactor comprising the steps of filling a rotatable perfusable culture chamber of the rotatable perfused TVEMF-bioreactor with a culture medium, placing cells in the rotatable perfusable culture chamber to initiate a three-dimensional TVEMF culture, rotating the rotatable perfusable culture chamber about an axis at a rotation speed, controlling the rotation of the rotatable perfusable culture chamber by adjusting the rotation speed to maintain the three-dimensional TVEMF culture, and exposing the cells to a TVEMF. The TVEMF is preferrably in the form of a delta wave, more preferably a differentiated square wave, and most preferably a square wave (following a Fourier curve). Preferably the method of the present invention has the properties of collocation of the culture medium and the cells, essentially no relative motion of the culture medium with respect to the rotatable perfusable culture chamber, and freedom for three-dimensional spatial orientation of the cells, while at the same time, maintaining essentially the same three-dimensional geometry, and cell-to-cell support and geometry as that of the cells found in vivo.

Other aspects, features, and advantages of the present invention will be apparent from the following description of the preferred embodiments of the invention given for the purpose of disclosure. This invention may be more fully described by the preferred embodiment(s) as hereinafter described, but is not intended to be limited thereto.

### BRIEF DESCRIPTION OF THE DRAWINGS

In the drawings,

FIG. 1 schematically illustrates a preferred embodiment of a culture medium flow loop;

FIG. 2 is a vertical cross-sectional view of a preferred embodiment of a rotatable perfused TVEMF-bioreactor;

FIG. 3 is a cross section along line 3-3 of FIG. 2;

FIG. 4 is a vertical cross sectional view of a preferred embodiment of a rotatable perfused TVEMF-bioreactor;

FIG. 5 is a vertical cross sectional view of a rotatable perfused TVEMF-bioreactor;

FIG. 6 is the orbital path of a typical cell in a non-rotating reference frame.

FIG. 7 is a graph of the magnitude of deviation of a cell per revolution.

FIG. 8 is a representative cell path as observed in a rotating reference frame of the culture medium.

FIG. 9 is a side view of a TVEMF-device.

FIG. 10 is an elevated front view of the TVEMF-device shown in FIG. 9.

FIG. 11 is an elevated front view of the TVEMF-device also shown in FIGS. 9 and 10 further showing a rotatable perfused TVEMF bioreactor adjacently located therein.

### DETAILED DESCRIPTION OF THE DRAWINGS

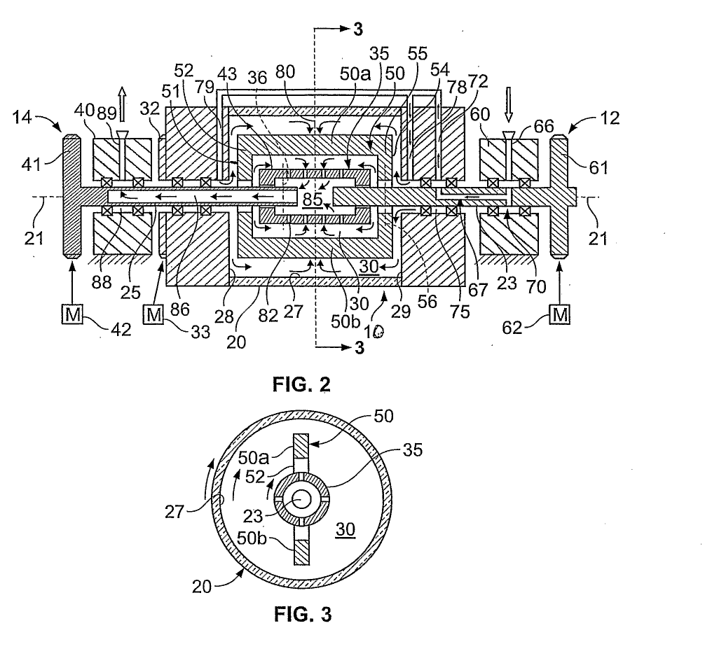

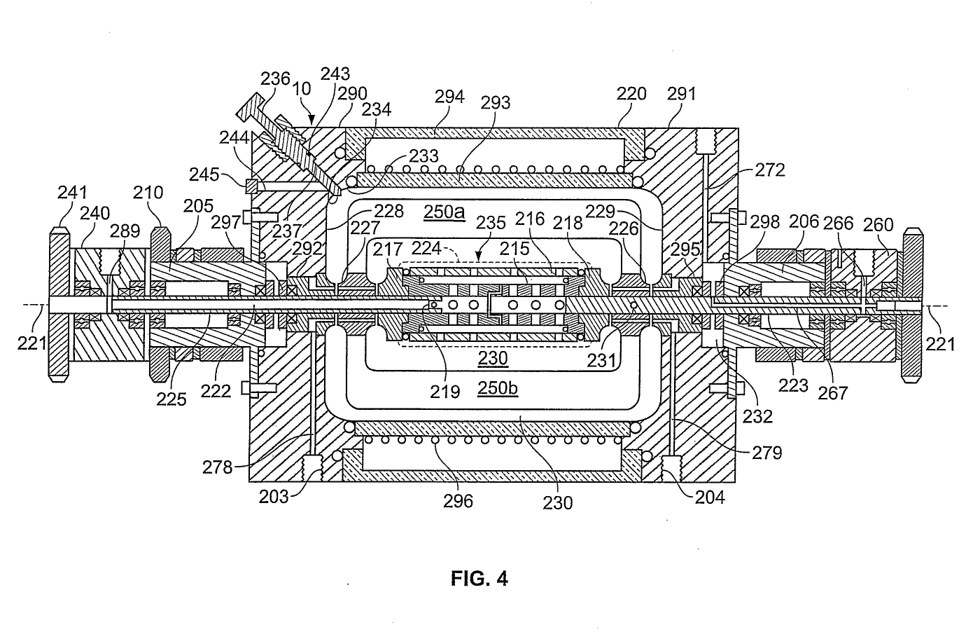

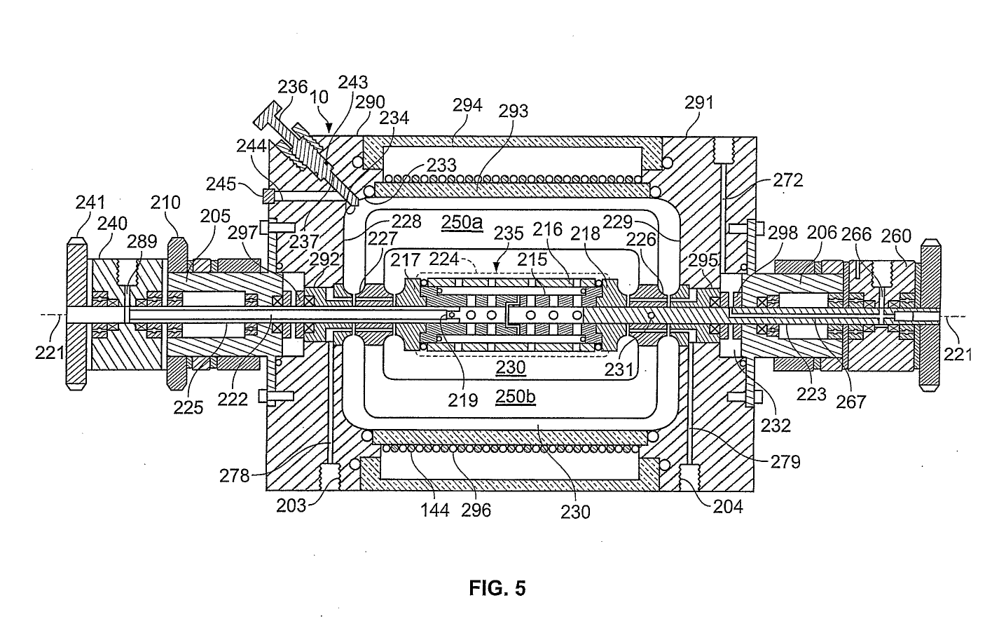

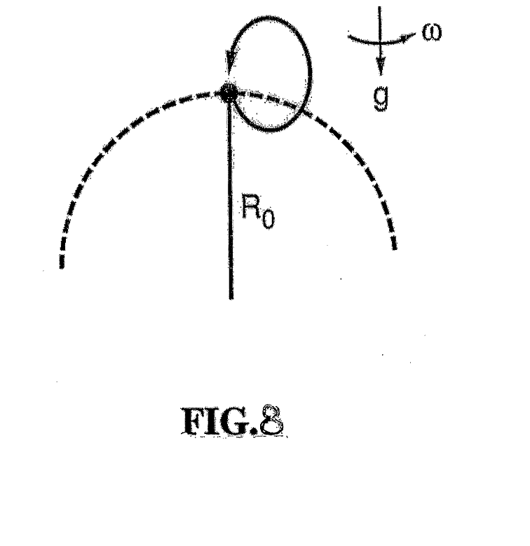

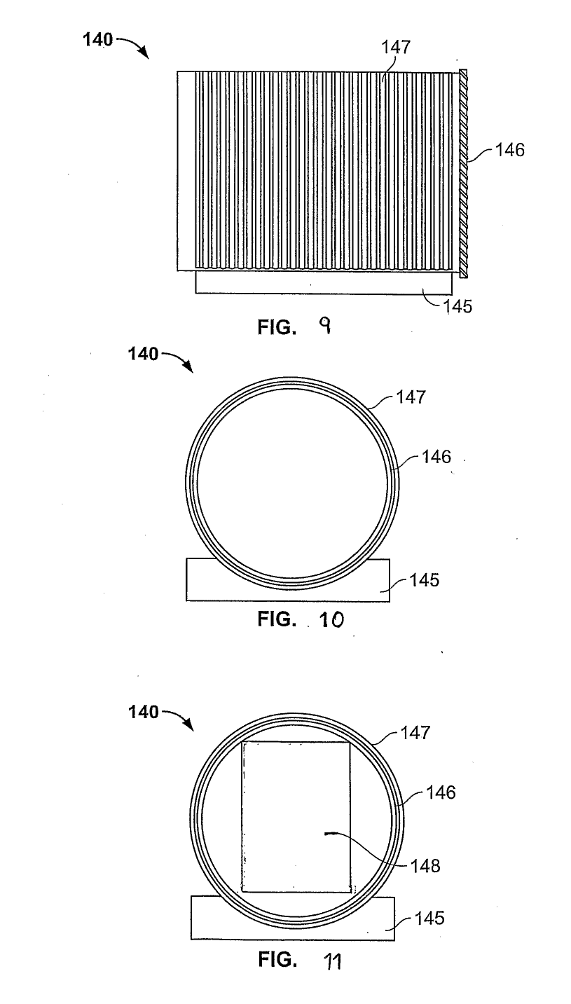

In the simplest terms, a rotatable perfused TVEMF-bioreactor comprises a rotatable perfusable culture chamber and a time varying electromagnetic force source (“TVEMF-source”) operatively connected to the rotatable perfusable culture chamber. Preferred rotatable perfused TVEMF-bioreactors are described herein. FIGS. 2 and 3 illustrate a preferred embodiment of a rotatable perfused TVEMF-bioreactor 10 with an adjacent TVEMF-source (not shown). FIG. 4 is a cross section of a rotatable perfused TVEMF-bioreactor 10 for use in the present invention in a preferred form with an integral TVEMF-source. FIG. 5 illustrates a rotatable perfused TVEMF-bioreactor 10 with an integral TVEMF-source. FIGS. 9-11 show an adjacent TVEMF-source of a rotatable perfused TVEMF-bioreactor.

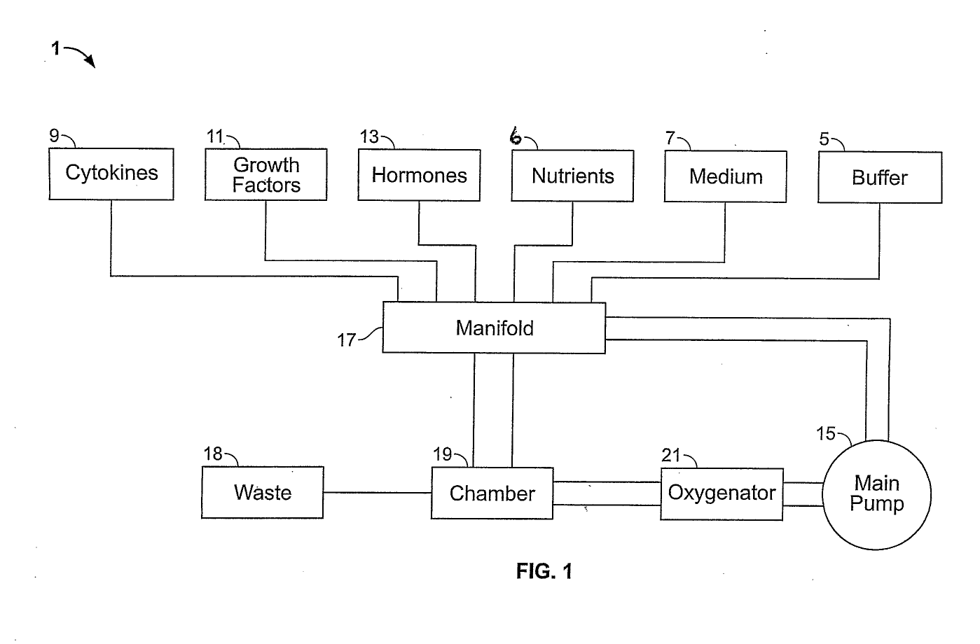

When in use, the present invention provides for the support of respiratory gas exchange in, supply of nutrients in, and removal of metabolic waste products from a three-dimensional TVEMF culture by the perfusion of culture medium through the rotatable perfusable culture chamber. The term “perfusable,” and similar terms, is intended to mean that the culture medium may be poured over, diffused or permeated through, cells in a perfusable rotatable culture chamber of a rotatable perfused TVEMF-bioreactor. The perfusion may preferably be through a culture medium flow loop as illustrated in FIG. 1, more preferably via direct injection, and most preferably via exchange across a diffusion membrane. In the drawings, referring now to FIG. 1, illustrated is a preferred embodiment of a culture medium flow loop 1 of a rotatable perfused TVEMF-bioreactor having a rotatable perfusable culture chamber 19, an oxygenator 21, an apparatus for facilitating the directional flow of the culture medium, preferably by the use of a main pump 15, and a supply manifold 17 for the selective input of culture medium requirements such as, but not limited to, nutrients 6, buffers 5, fresh medium 7, cytokines 9, growth factors 11, and hormones 13. In this preferred embodiment, the main pump 15 provides fresh medium from the supply manifold 17 to the oxygenator 21 where the medium is oxygenated and passed through the rotatable perfusable culture chamber 19. The waste in the spent medium from the rotatable perfusable culture chamber 19 is removed, preferably by the main pump 15, and delivered to the waste 18 and the remaining cell culture medium not removed to the waste 18 is returned to the manifold 17 where it may preferably receive a fresh charge of culture medium requirements before recycling by the pump 15 through the oxygenator 21 to the rotatable perfusable culture chamber 19.

In operation, the culture medium is circulated through the three-dimensional TVEMF culture in the rotatable perfusable culture chamber 19 and preferably around the culture medium flow loop 1, as shown in FIG. 1. In this preferred embodiment of a culture medium flow loop 1, adjustments are made in response to chemical sensors (not shown) that maintain constant conditions within the rotatable perfusable culture chamber 19. Controlling carbon dioxide pressures and introducing acids or bases corrects pH. Oxygen, nitrogen, and carbon dioxide are dissolved in a gas exchange system (not shown) in order to support cell respiration. The culture medium flow loop 1 adds oxygen and removes carbon dioxide from a circulating gas capacitance. Although FIG. 1 is one preferred embodiment of a culture medium flow loop that may be used in the present invention, the invention is not intended to be so limited. The input of culture medium requirements such as, but not limited to, oxygen, nutrients, buffers, fresh medium, cytokines, growth factors, and hormones into a rotatable perfused TVEMF-bioreactor can also be performed manually, automatically, or by other control means, as can be the control and removal of waste and carbon dioxide.

FIG. 2 is a cross-sectional side view of a preferred embodiment of a rotatable perfused TVEMF-bioreactor 10 that is operatively connected to an adjacent TVEMF-source (not shown) having an input end 12 and an output end 14. In FIG. 2, an outer tubular housing 20 rotatably supported for rotation about a horizontal central axis 21 and about an input shaft 23 and an output shaft 25 which are aligned with the central axis 21 (not shown except by dashed line). The outer tubular housing 20 has an interior wall 27, preferably cylindrically shaped, an output transverse end wall 28, and an input traverse end wall 29 that generally define a rotatable perfusable culture chamber 30, preferably cylindrically shaped, preferably elongated. A spur gear 32 is attached to one end of the outer tubular housing 20 and is driven by a motor 33 to rotate the housing 20 about its horizontal central axis 21.

Coaxially disposed about the central axis 21 is an inner filter assembly 35, preferably tubular, that is rotatably mounted on the input shaft 23 and is coupled (as shown by the dashed line 36) to the output shaft 25. The output shaft 25, in turn, is rotatably supported in an output stationary housing 40 and the output shaft 25 has an externally located output spur gear 41 that is connected to a first independent drive motor 42 for rotating the output shaft 25 and the inner filter assembly 35 independently of the outer housing 20. The annular space between the inner filter assembly 35 and the interior wall 27 of the outer tubular housing 20 define the rotatable perfusable culture chamber 30 located about the horizontal axis 21. Intermediate of the outer wall 43 of the inner filter assembly 35 and the interior wall 27 of the outer housing 20 is an intermediate blade member system 50 which preferably includes two lengthwise extending blade members 50a and 50b which are preferably equiangularly spaced from one another about the central axis 21. Each of the blade members 50a and 50b at a first longitudinal end 51 has a second radial arm 52 which is rotatably supported on the output shaft 25 and at a second longitudinal end 54 has a second radial arm 55 which is coupled to the input shaft 23 (shown by dashed line 56). The input shaft 23, in turn, is rotatably mounted in an input stationary housing 60 and the input shaft 23 has an input spur gear 61 that is driven by a second independent drive motor 62 for rotation of the blade member system 50 independent of the rotation of the outer housing 20.

As shown in FIG. 3, the angular rotation of the three sub-assemblies 20, 35 and 50, i.e., the inner filter assembly or member 35, the outer housing 20, and the intermediate blade member system 50, may preferably be at the same angular rate and in the same direction about a horizontal rotational axis and preferably substantially in the same direction about a horizontal axis so that there is substantially no relative movement between the three sub-assemblies. This condition of operation obtains a clinostat suspension of microcarrier beads in a fluid medium within the rotatable perfusable culture chamber 30 of the rotatable perfused TVEMF-bioreactor 10 without turbulence.

The rotation of the filter 35 can preferably be started and stopped to, for instance, add culture medium, which will cause turbulence on the surface of the filter 35 and keep the surface clean. The blade members 50a and 50b assist cells as they grow to maintain spatial positions in the rotating culture medium. This is particularly helpful for higher density cells, tissues, and tissue-like structures, for instance, bone cells. By rotating the fluid and the outer housing 20, the velocity gradient at the wall boundary layer is nearly eliminated.

The rotatable perfused TVEMF-bioreactor 10 of FIG. 2, in operation provides for culture medium preferably containing fresh nutrients and gases to be input to an input passageway 66 in the input stationary housing 60 and connects to an input longitudinal passageway 67 in the input shaft 23 by virtue of a sealed input rotative coupling 70. The input passageway 67 in the input shaft 23 couples to a radial supply passageway 72 in an end cap of the outer housing 20 by virtue of a sealed input rotative coupling 75. The radial supply passageway 72, in turn, connects to space apart a radially directed input end input passage 78 and output end input passage 79 in the outer housing 20 where the input end input passage 78, and output end input passage 79 are located at opposite ends of the rotatable perfusable culture chamber 30. As shown by the arrows, when medium is input at both ends of the rotatable perfusable culture chamber 30, the medium moves radially outward toward the interior wall 27 of the outer housing 20 and then moves longitudinally in a horizontal direction toward a midpoint plane generally indicated by a vertically dashed line 80 and then moves radially inwardly toward the outer wall 43 of inner filter assembly 35. Thus the medium in the chamber 30 has a generally toroidal type of motion in radial planes on either side of the midpoint plane 80 of the outer housing 20. The inner filter assembly 35 has apertures 82 along its length for exit of culture medium from the rotatable perfusable culture chamber 30 to the interior and, while not illustrated in FIG. 2, preferably there is a lengthwise extending filter cloth located across the apertures 82 that prevents microcarrier bead members in the chamber 30 from exiting through the apertures 82. Spent culture medium in the rotatable perfusable culture chamber 30 thus is passed to the interior 85 of the inner filter assembly 35 and exits via an output longitudinal passageway 86 in the output shaft 25 to an output rotative coupling output 88 in the output stationary housing 40 and to an output passageway 89 preferably to the return of the culture medium flow loop for recharging (not shown).

Turning now to the preferred embodiment of a rotatable perfused TVEMF-bioreactor 10 comprising a rotatable perfusable culture chamber 230 and a TVEMF-source operatively connected to the rotatable perfusable culture chamber 230, wherein the TVEMF-source is an annular wire heater 296. The annular wire heater 296 is integral with the rotatable perfusable culture chamber 230. The rotatable perfusable culture chamber 230 is preferably transparent, and further comprising an outer housing 220 which includes a first 290 and second 291 transverse end cap member, preferably cylindrically shaped, having facing first 228 and second 229 end surfaces arranged to receive an inner member 293, preferably cylindrical tubular, preferably transparent, and more preferably glass, and an outer tubular member 294, preferably transparent, and preferably glass. Suitable pressure seals are well known in the art and are preferably provided. Between the inner 293 and outer 294 tubular members is an annular wire heater 296 that can preferably be utilized for obtaining the proper incubation temperatures for TVEMF-expansion. The wire heater 296 can also preferably be used as a TVEMF-source that, in use, supplies a TVEMF to the rotatable perfusable culture chamber 230. The first end cap member 290 and second end cap member 291 have inner curved surfaces adjoining the end surfaces 228, 229 for promoting smoother flow of the mixture within the culture chamber 230. The first end cap member 290, and second end cap member 291 have a first central fluid transfer journal member 292 and second central fluid transfer journal member 295, respectively, that are rotatably received respectively on an input shaft 223 and an output shaft 225. Each transfer journal member 294, 295 has a flange to seat in a recessed counter bore in an end cap member 290, 291 and is attached by a first lock washer and ring 297, and second lock washer and ring 298 against longitudinal motion relative to a shaft 223, 225. Each journal member 294, 295 has an intermediate annular recess that is connected to longitudinally extending, circumferentially arranged passages. Each annular recess in a journal member 292, 295 is coupled by a first radially disposed passage 278 and second radially disposed passage 279 in an end cap member 290 and 291, respectively, to first input coupling 203 and second input coupling 204. In operation, culture medium in a radial passage 278 or 279 flows through a first annular recess and the longitudinal passages in a journal member 292 or 295 to permit access to the medium through a journal member 292, 295 to each end of the journal member 292, 295 where preferably the access is circumferential about a shaft 223, 225.

Attached to the end cap members 290 and 291 are a first tubular bearing housing 205, and second tubular bearing housing 206 containing ball bearings which relatively support the outer housing 220 on the input 223 and output 225 shafts. The first bearing housing 205 has an attached first sprocket gear 210 for providing a rotative drive for the outer housing 220 in a rotative direction about the input 223 and output 225 shafts and the longitudinal axis 221. The first bearing housing 205, and second bearing housing 206 also preferably have provisions for electrical take out of the annular wire heater 296 and preferably any other sensor, for instance a sensor to detect a change in the location of the cells, preferably and/or a sensor to detect a change in the pH and/or the temperature of the three-dimensional TVEMF culture.

The inner filter assembly 235 includes inner 215 and outer 216 tubular members having perforations and/or apertures along their lengths and have a first 217 and second 218 inner filter assembly end cap member with perforations. The inner tubular member 215 is preferably constructed in two pieces with an interlocking centrally located coupling section and each piece attached to an end cap 217 or 218. The outer tubular member 216 is preferably mounted between the first 217 and second inner filter assembly end caps.

The end cap members 217, 218 are respectively rotatably supported on the input shaft 223 and the output shaft 225. The inner member 215 is rotatively attached to the output shaft 225 preferably by a pin and an interfitting groove 219. A cloth 224, preferably nylon, with a weave, preferably ten-microns, is disposed over the outer surface of the outer member 216 and is attached at either end, preferably with O-rings. Because the inner member 215 is attached to a slot in the output drive shaft 225, preferably by a coupling pin, the output drive shaft 225 can rotate the inner member 215. The inner member 215 is coupled by the first 217 and second 218 end caps that support the outer member 216. The output shaft 225 is extended through bearings in a first stationary housing 240 and is coupled to a first sprocket gear 241. As illustrated, the output shaft 225 has a tubular bore 222 that extends from a first passageway 289 in the first stationary housing 240 located between seals to the inner member 215 so that, in use, a flow of culture medium can be exited from the inner member 215 through the stationary housing 240.

Between the first 217 and second 218 end caps for the inner member 235 and the journals 292, 295 in the outer housing 220, are a first 227 and second 226 hub for the blade members 250a and 250b. The second hub 226 on the input shaft 223 is coupled to the input shaft 223 by a pin 231 so that the second hub 226 preferably rotates with the input shaft 223. Each hub 227, 226 has axially extending passageways for the transmittal of culture medium through a hub.

The input shaft 223 extends through bearings in the second stationary housing 260 for rotatable support of the input shaft 223. A second longitudinal passageway 267 extends through the input shaft 223 to a location intermediate of retaining washers and rings that are disposed in a second annular recess 232 between the faceplate and the housing 260. A third radial passageway 272 in the second end cap member 291 permits culture medium in the recess to exit from the second end cap member 291. While not shown, the third passageway 272 connects to each of the passages 278 and 279, preferably through piping and a Y joint.

A sample port is shown in FIG. 4, where a first bore 237 extending along a first axis intersects a corner 233 of the chamber 230 and forms a restricted aperture 234. Preferably the bore 237 has a counter bore and a threaded ring at one end to receive a cylindrical valve member 236, preferably threadedly. The valve member 236 protrudes slightly into the interior of the chamber 230, and the valve member 236 comprises a complimentarily formed tip to engage the opening 234 and. Preferably, an O-ring 243 on the valve member 236 provides a seal. A second bore 244 along a second axis intersects the first bore 237 at a location between the O-ring 243 and the opening 234. Preferably an elastomer or plastic stopper 245 closes the second bore 244 that can be entered with a syringe for removing a sample. To remove a sample, the valve member 236 is backed off to access the opening 234 and the bore 244. A syringe can then be used to extract a sample and the opening 234 can be reclosed, and therefore, no outside contamination reaches the interior of the TVEMF-bioreactor 10.

In operation, culture medium is input to the second port or passageway 266 to the shaft passageway and thence to the first radially disposed 278 and second radially disposed passageways 279 via the third radial passageway 272. In operation, when the culture medium enters the chamber 230 via the longitudinal passages in the journals 292, 294 the culture medium impinges on an end surface 228, 229 of the hubs 227, 226 and is dispersed radially as well as axially through the passageways in the hubs 227, 226. Culture medium passing through the hubs 227, 226 impinges on the end cap members 217, 218 and is dispersed radially. The flow of entry culture medium is thus radially outward away from the longitudinal axis 221 and flows in a toroidal fashion from each end to exit through the polyester cloth 224 and openings in the filter assembly 235 to exit via the passageways 266 and 289. By controlling the rotational speed and direction of rotation of the outer housing 220, chamber 230, and inner filter assembly 235 any desired type of culture medium action can be obtained. Preferably a substantially clinostat operation can be obtained together with a continuous supply of culture medium.

FIG. 5 is a cross-sectional elevated side view of the preferred embodiment of a rotatable perfused TVEMF bioreactor 10. The preferred embodiment of a rotatable perfused TVEMF-bioreactor 10, illustrated in FIG. 5, shows a wire coil 144, which is a TVEMF-source, that is integral with the rotatable perfused TVEMF-bioreactor 10, but is separate from the annular wire heater 296, both of which can be used as a TVEMF-source. The preferred embodiment of a rotatable perfused TVEMF bioreactor 10 illustrated in FIG. 5 is different from the preferred embodiment of a rotatable perfused TVEMF-bioreactor illustrated in FIG. 4 because FIG. 4 only discloses an annular wire heater 296 that can preferably be used as a TVEMF-source.

If a TVEMF is not applied using an integral TVEMF-source for instance an annular wire heater 296, as in FIG. 4, or an integral TVEMF-source for instance a wire coil 144 as in FIG. 5, it can be supplied by another preferred TVEMF-source. For instance, FIGS. 9-11 illustrate a preferred embodiment of another TVEMF-source, a TVEMF-device 140 that may preferably supply a TVEMF to a three-dimensional TVEMF culture in a rotatable perfused TVEMF-bioreactor which does not have an integral TVEMF-source, for example the rotatable perfused TVEMF-bioreactor illustrated in FIG. 2. Specifically, FIG. 9 is a preferred embodiment of a TVEMF-device 140 that may be operatively connected to a rotatable perfusable culture chamber. FIG. 9 is an elevated side perspective of the TVEMF-device 140 which comprises a support base 145, a coil support 146 disposed on the base 145 with a wire coil 147 wrapped around the support 146. FIG. 10 is an elevated front perspective of a TVEMF-device 140 illustrated in FIG. 9. FIG. 11 illustrates the TVEMF-device operatively connected to a rotatable perfused TVEMF-bioreactor 148, such as that of FIG. 2, so that the rotatable perfused TVEMF-bioreactor 148 in FIG. 11 has an adjacent TVEMF-source. In use, a rotatable perfused TVEMF-bioreactor 148 may preferably be inserted into the coil support 146 which is disposed on a support base 145 and which is wound by a wire coil 147. Since the TVEMF-device 140 is adjacent to the rotatable perfused TVEMF-bioreactor 148, the TVEMF-device 140 can be reused as a TVEMF-source. In addition, since the TVEMF-device 140 is adjacent to the rotatable perfused TVEMF-bioreactor 148, the TVEMF-device 140 can be used to generate a TVEMF in all types of TVEMF-bioreactors, preferably rotatable.

As various changes could be made in rotatable perfused TVEMF-bioreactors subjected to a time varying electromagnetic force as are contemplated in the present invention, without departing from the scope of the invention, it is intended that all matter contained herein be interpreted as illustrative and not limiting.

### DETAILED DESCRIPTION OF THE INVENTION

The following definitions are meant to aid in the description and understanding of the defined terms in the context of the present invention. The definitions are not meant to limit these terms to less than is described throughout this application. Furthermore, several definitions are included relating to TVEMF—all of the definitions in this regard should be considered to complement each other, and not construed against each other.

As used throughout this application, the term “TVEMF” refers to “time varying electromagnetic force”. As discussed above, the TVEMF of this invention is in a delta wave, more preferably a differential square wave, and most preferably a square wave (following a Fourier curve). Preferably, the square wave has a frequency of about 2 to about 25 cycles/second, more preferably about 5 to about 20 cycles/second, and for example about 10 cycles/second, and the TVEMF-source has an RMS value preferably of from about 0.1 mA to about 1000 mA, more preferably about 1 mA to about 10 mA, for instance 6 mA. The TVEMF applied to a rotatable perfused TVEMF-bioreactor is preferably in the range of from about 0.05 to about 6.0 gauss, more preferably of from about 0.05 to about 0.5 gauss, and most preferably about 0.5 gauss. However, these parameters are not meant to be limiting to the TVEMF of the present invention, as such may vary based on other aspects of this invention. TVEMF may be measured for instance by standard equipment such as an EN131 Cell Sensor Gauss Meter.

As used throughout this application, the term “rotatable perfused TVEMF-bioreactor” is meant to comprise a TVEMF-source operatively connected to a rotatable perfusable culture chamber. In use, a rotatable perfused TVEMF-bioreactor refers to a bioreactor to which TVEMF is applied, that may be rotated and that provides a method for sustaining the three-dimensional TVEMF culture by perfusion, as described for instance in the Description of the Drawings, above. In operation, the TVEMF, at an appropriate gauss level, is delivered to the interior portion of the rotatable perfusable culture chamber. The volume of the rotatable perfusable culture chamber is preferably of from about 100 ml to about 3 liters. See for instance FIGS. 2, 3, 4 and 5 herein for examples (not meant to be limiting) of a rotatable perfused TVEMF-bioreactor of the present invention. Preferably, a rotatable perfused TVEMF-bioreactor allows for the exchange of growth medium (preferably with additives) and for oxygenation of the three-dimensional TVEMF culture. Without being bound by theory, the rotatable perfused TVEMF-bioreactor provides for the TVEMF-expansion of cells for several days or more.

As used throughout this application, the term “three-dimensional TVEMF culture” and similar terms, refers to a mixture of cells with culture medium that allows the cells to expand, for TVEMF-expansion of the cells in a rotatable perfusable culture chamber. The culture medium and the cells, tissue, or tissue-like structure in combination is referred to as a three-dimensional TVEMF culture when located in the rotatable perfusable culture chamber of the rotatable perfused TVEMF-bioreactor, and/or after TVEMF has been delivered thereto. The three-dimensional TVEMF-culture is preferably supported by TVEMF-expansion in a rotatable perfused TVEMF-bioreactor wherein cells maintain their three-dimensional geometry, and cell-to-cell support and geometry. Three-dimensional tissue and/or tissue-like structures can also develop from the cells and be sustained and further expanded in the three-dimensional TVEMF culture.

As used throughout this application, the term “operatively connected,” and similar terms, is intended to mean that the TVEMF-source can be connected to the rotatable perfusable culture chamber in a manner such that when in operation, the TVEMF-source can impart a TVEMF to the rotatable perfusable culture chamber and the cells, tissue, or tissue-like structures contained therein. The TVEMF-source may be operatively connected if it is integral with the rotatable perfusable culture chamber, and may also be operatively connected if it is adjacently located to the rotatable perfusable culture chamber. The TVEMF-source is operatively connected if it can impart a TVEMF to the inside of the rotatable perfusable culture chamber.

As used throughout this application, the term “exposing,” and similar terms, refers to the process of supplying a TVEMF to cells contained in a rotatable perfusable culture chamber. In operation, the present invention provides that the TVEMF-source is turned on and set at a preferred gauss range and a preferred wave form so that the same is delivered via the TVEMF-source, preferably a wire coil, more preferably an annular wire heater, most preferably a TVEMF-device. The TVEMF is then delivered through the TVEMF-source to the cells in the rotatable perfusable culture chamber thus exposing the cells to the TVEMF.

As used throughout this application, the term “cells” refers to a cell in any form, for example, individual cells, tissue, cell aggregates, cells pre-attached to cell attachment substrates for instance microcarrier beads, tissue-like structures, or intact tissue resections. The cells may be from, but are not limited to, the following sources: mammalian, reptilian, avian, from fish, from yeast, and bacterial.

As used throughout this application, the term “culture medium” and similar terms, refers to a liquid comprising, but not limited to, culture media and nutrients, which is meant for the sustenance of cells over time. The culture medium may be enriched with any of the following, but is not limited thereto: culture media, buffer, growth factors, hormones, and cytokines. The culture medium is supplied to the cells for suspension therein and to support TVEMF-expansion. The culture medium may preferably be mixed with the cells before it is added to the rotatable perfusable culture chamber of the rotatable perfused TVEMF-bioreactor, or may preferably be added before the cells are added to the rotatable perfusable culture chamber thereby mixing the culture medium and the cells in the rotatable perfusable culture chamber. The culture medium and the cells combination is referred to as a three-dimensional TVEMF culture when located in the rotatable perfused TVEMF-bioreactor, and/or after TVEMF has been delivered thereto. The culture medium can preferably be enriched and/or refreshed during TVEMF-expansion as needed. Waste contained in culture medium, as well as culture medium itself, can be removed from the three-dimensional TVEMF culture as needed. Waste contained in the spent culture medium can be, but is not limited to, metabolic waste, dead cells, and other toxic debris. The culture medium can preferably be enriched with oxygen and preferably has oxygen, carbon dioxide, and nitrogen carrying capabilities.

As used throughout this applications, the term, “placing,” and similar terms, refers to the process of suspending cells in culture medium before adding the combination cells and culture medium to the rotatable perfusable culture chamber of the rotatable perfused TVEMF-bioreactor. The term “placing,” may also refer to adding cells to culture medium that is already present in the rotatable perfusable culture chamber. The cells may be suspended in additional preferred liquids including, for instance, PBS and/or plasma. The culture medium and the cells combination is referred to as a three-dimensional TVEMF culture when located in the rotatable perfused TVEMF-bioreactor, during and after TVEMF has been delivered thereto. Cells can be placed into the rotatable perfusable culture chamber along with cell attachment substrates and also with

As used throughout this application, the term “TVEMF-expansion,” and similar terms, refers to the process of increasing the number of cells in a rotatable perfused TVEMF-bioreactor, by subjecting the cells to a TVEMF of from about 0.05 to about 6.0 gauss. Preferably the cells are TVEMF-expanded to more than seven times their original number. The expansion of cells in a rotatable perfused TVEMF-bioreactor according to the present invention provides for cells that maintain, or have the same or essentially the same, three-dimensional geometry and cell-to-cell support and cell-to-cell geometry as the cells prior to TVEMF-expansion. Other aspects of TVEMF-expansion may also provide the exceptional characteristics of the cells of the present invention. Not to be bound by theory, TVEMF-expansion not only provides for high concentrations of cells that maintain their three-dimensional geometry and cell-to-cell support, but also supports and maintains the growth of three-dimensional tissues and tissue-like structures.

As used throughout this application, the term “cell-to-cell geometry” refers to the geometry of cells including the spacing, distance between, and physical relationship of the cells relative to one another. For instance, TVEMF-expanded cells, including those of tissues, cell aggregates, and tissue-like structures, of this invention stay in relation to each other as in the body. The expanded cells are within the bounds of natural spacing between cells, in contrast to for instance two-dimensional expansion containers, where such spacing is not preserved over time and TVEMF-expansion.

As used throughout this application, the term “cell-to-cell support” refers to the support one cell provides to an adjacent cell. For instance, tissues, cell aggregates, tissue-like structures, and cells maintain interactions such as chemical, hormonal, neural (where applicable/appropriate) with other cells in the body. In the present invention, these interactions are maintained within normal functioning parameters, meaning they do not for instance begin to send toxic or damaging signals to other cells (unless such would be done in the natural cellular and tissue environment).

As used throughout this application, the term “three-dimensional geometry” refers to the geometry of cells in a three-dimensional state (same as or very similar to their natural state), as opposed to two-dimensional geometry for instance as found in cells grown in a Petri dish, where the cells become flattened and/or stretched. Not to be bound by theory, but the three-dimensional geometry of the cells is maintained, supported, and preserved such that the cell can develop into three-dimensional cell aggregates, tissues and/or tissue-like structures in the three-dimensional TVEMF culture of the rotatable perfused TVEMF-bioreactor. Furthermore, tissues can also be cultured in the rotatable perfused TVEMF-bioreactor, while at the same time, maintaining the three-dimensional geometry, and cell-to-cell support and geometry.

For each of the above three definitions, relating to maintenance of “cell-to-cell support” and “cell-to-cell geometry” and “three-dimensional geometry” of the cells of the present invention, the term “essentially the same” means that natural geometry and support are provided in TVEMF-expansion, so that the cells are not changed in such a way as to be for instance dysfunctional, toxic or harmful to other cells.

In operation, cells are placed into the rotatable perfusable culture chamber of the rotatable perfused TVEMF-bioreactor. The rotatable perfusable culture chamber is rotated over a period of time during which a TVEMF is generated in the rotatable perfusable culture chamber by the TVEMF-source. By “during which,” it is intended that the initiation of the delivery of the TVEMF may be before, concurrent with, or after rotation of the rotatable perfusable culture chamber is initiated. Upon completion of the period of time, the TVEMF-expanded cells are removed from the rotatable perfusable culture chamber. In a more complex rotatable perfused TVEMF-bioreactor, a culture medium enriched with culture medium requirements preferably including, but not limited to, cell culture media, buffer, nutrients, hormones, cytokines, and growth factors, which provides sustenance to the cells, can be periodically refreshed and removed.

In use, the present invention provides a stabilized culture environment into which cells may be introduced, suspended, assembled, grown, and maintained with improved retention of delicate three-dimensional structural integrity by simultaneously minimizing the fluid shear stress, providing three-dimensional freedom for cell and substrate spatial orientation, and increasing localization of cells in a particular spatial region for the duration of the culture. In use, the present invention provides these three criteria (hereinafter referred to as “the three criteria above”), and at the same time, has a TVEMF-source that exposes the cells to a TVEMF supplied by a TVEMF-source, preferably in a square wave, more preferably a differential square wave, most preferably a delta wave, to the cells so that TVEMF-expansion of the cells is enhanced. Of particular interest to the present invention is the dimension of the rotatable perfusable culture chamber, the sedimentation rate of the cells, the rotation rate, the external gravitational field, and the TVEMF.

The stabilized culture environment referred to in the operation of present invention is that condition in the culture medium, particularly the fluid velocity gradients, prior to introduction of cells, which will support a nearly uniform suspension of cells upon their introduction thereby creating a three-dimensional TVEMF culture upon addition of the cells. In a preferred embodiment, the culture medium is initially stabilized into a near solid body horizontal rotation about an axis within the confines of a similarly rotating chamber wall of a rotatable perfused TVEMF-bioreactor. The chamber walls are set in motion relative to the culture medium and internal chamber components so as to initially introduce essentially no fluid stress shear field therein. Cells are introduced to, and move through, the culture medium in the stabilized culture environment and a TVEMF is applied thereto thus creating a three-dimensional TVEMF culture. The cells move under the influence of gravity, centrifugal, and coriolus forces, and the presence of cells within the culture medium of the three-dimensional TVEMF culture induces secondary effects to the culture medium. The significant motion of the culture medium with respect to the rotatable perfusable culture chamber walls, significant fluid shear stress, and other fluid motions, is due to the presence of these cells within the culture medium.

In most cases the cells with which the stabilized culture environment is primed sediment at a slow rate preferably under 0.1 centimeter per second. It is therefore possible, at this early stage of the three-dimensional TVEMF culture, to select from a broad range of rotational rates (preferably of from about 5 to about 120 RPM) and chamber diameters (preferably of from about 0.5 to about 36 inches). Preferably, the slowest rotational rate is advantageous because it minimizes equipment wear and other logistics associated with handling of the three-dimensional TVEMF culture.

Not to be bound by theory, rotation about a substantially horizontal axis with respect to the external gravity vector at an angular rate optimizes the orbital path of cells suspended within the three-dimensional TVEMF culture. In operation, the cells expand to form a mass of cell aggregates, three-dimensional tissues, and/or tissue-like structures, which increase in size as the three-dimensional TVEMF culture progresses. The progress of the three-dimensional TVEMF culture is preferably assessed by a visual, manual, or automatic determination of an increase in the diameter of the three-dimensional tissue mass in the three-dimensional TVEMF culture. An increase in the size of the cell aggregate, tissue, or tissue-like structure in the three-dimensional TVEMF culture may require appropriate adjustment of rotational rates in order to optimize the particular paths. The rotation of the rotatable perfusable culture chamber optimally controls collision frequencies, collision intensities, and localization of the cells in relation to other cells and also the limiting boundaries of the rotatable perfusable culture chamber. If the cells are observed to excessively distort inwards on the downward side and outwards on the upwards side then RPM may preferably be increased. If the cells are observed to centrifugate excessively to the outer walls then the RPM may preferably be reduced. Not to be bound by theory, as the operating limits are reached, in terms of high cell sedimentation rates or high gravity strengths, the operator may be unable to satisfy both of these conditions and may be forced to accept degradation in performance as measured against the three criteria above.

The cell sedimentation rate and the external gravitations field place a lower limit on the fluid shear stress obtainable, even within the operating range of the present invention, due to gravitationally induced drift of the cells through the culture medium of the three-dimensional TVEMF culture. Calculations and measurements place this minimum fluid shear stress very nearly to that resulting from the cells' terminal sedimentation velocity (through the culture medium) for the external gravity field strength. Centrifugal and coriolis induced motion [classical angular kinematics provide the following equation relating the Coriolis force to an object's mass (in), its velocity in a rotating frame (vᵣ) and the angular velocity of the rotating frame of reference ( [symbol] ): F_{Coriolis}=−2 m (w×vᵣ)] along with secondary effects due to cell and culture medium interactions, act to further degrade the fluid shear stress level as the cells, cell aggregates, tissues, and tissue-like structures increase in size.

Not to be bound by theory, but as the external gravity field is reduced much denser and larger three-dimensional structures can be obtained. In order to obtain the minimal fluid shear stress level it is preferable that the chamber walls and internal components be rotated at substantially the same rate as the culture medium. Not to be bound by theory, but this minimizes the fluid velocity gradient induced upon the three-dimensional TVEMF culture. Preferably, selected levels of fluid shear stress may be introduced to the three-dimensional TVEMF culture by differential rotation of chamber components. It is advantageous to control the rate and size of tissue formation in order to maintain the cell size (and associated sedimentation rate) within a range for which the process is able to satisfy the three criteria above. However, preferably, the velocity gradient and resulting fluid shear stress may be intentionally introduced and controlled for specific research purposes such as studying the effects of shear stress on the three-dimensional tissue. In addition, transient disruptions of the stabilized culture environment are permitted and tolerated for, among other reasons, logistical purposes during initial system priming, sample acquisition, system maintenance, and culture termination.

Rotating cells about an axis substantially perpendicular to gravity can produce a variety of sedimentation rates, all of which according to the present invention remain spatially localized in distinct regions for extended periods of time ranging from seconds (when sedimentation characteristics are large) to hours (when sedimentation differences are small). Not to be bound by theory, but this allows these cells, cell aggregates, tissues, and tissue-like structures sufficient time to interact as necessary to form multi-cellular structures and to associate with each other in a three-dimensional TVEMF culture. Preferably, cells undergo TVEMF-expansion preferably for at least 4 days, more preferably from about 7 days to about 14 days, most preferably from about 7 days to about 10 days, even more preferably about 7 days. Preferably, TVEMF-expansion may continue in a rotatable perfused TVEMF-bioreactor of the present invention for up to 160 days, or preferably at a rate of expansion that is about 1000 times the original concentration of cells.

Rotatable perfusable culture chamber dimensions also influence the path of cells in the three-dimensional TVEMF culture of the present invention. A rotatable perfusable culture chamber diameter is preferably chosen which has the appropriate volume, preferably of from about 100 ml to about 3 L, for the intended three-dimensional TVEMF culture and which will allow a sufficient seeding density of cells. Not to be bound by theory, but the outward cells drift due to centrifugal force is exaggerated at higher rotatable perfusable culture chamber radii and for rapidly sedimenting cells. Thus limiting the maximum radius of the rotatable perfusable culture chamber as a function of the sedimentation properties of the tissues anticipated in the final three-dimensional TVEMF culture stages (when large tissues with high rates of sedimentation have formed).

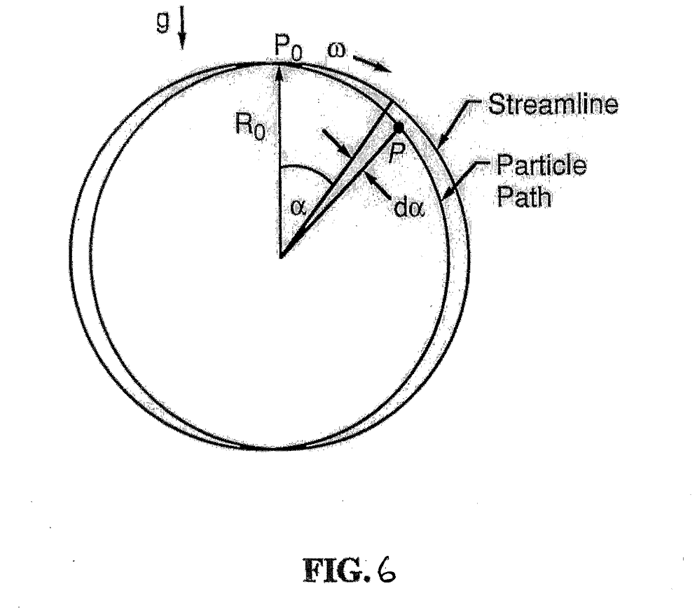

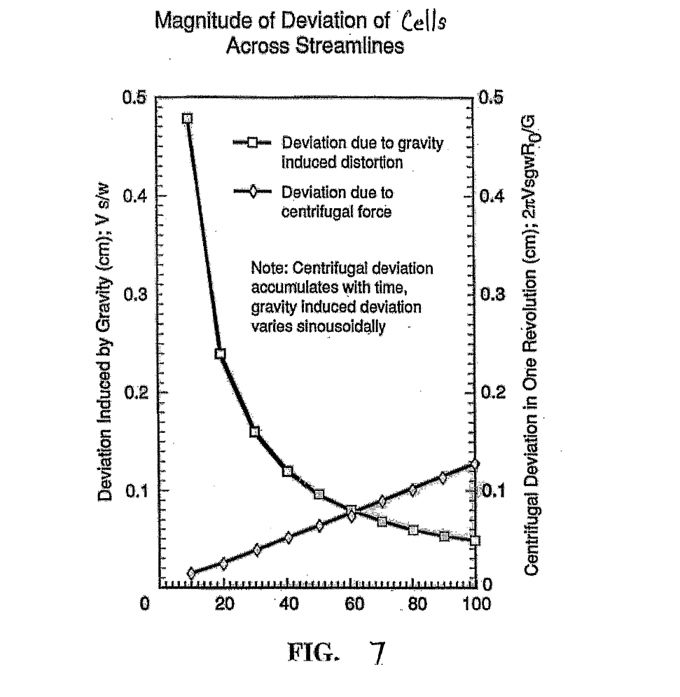

The path of the cells in the three-dimensional TVEMF culture has been analytically calculated incorporating the cell motion resulting from gravity, centrifugation, and coriolus effects. A computer simulation of these governing equations allows the operator to model the process and select parameters acceptable (or optimal) for the particular planned three-dimensional TVEMF culture. FIG. 6 shows the typical shape of the cell orbit as observed from the external (non-rotating) reference frame. FIG. 7 is a graph of the radial deviation of a cell from the ideal circular streamline plotted as a function of RPM (for a typical cell sedimenting at 0.5 cm per second terminal velocity). This graph (FIG. 7) shows the decreasing amplitude of the sinusoidally varying radial cells deviation as induced by gravitational sedimentation. FIG. 7 also shows increasing radial cells deviation (per revolution) due to centrifugation as RPM is increased. These opposing constraints influence carefully choosing the optimal RPM to preferably minimize cell impact with, or accumulation at, the chamber walls. A family of curves is generated which is increasingly restrictive, in terms of workable RPM selections, as the external gravity field strength is increased or the cell sedimentation rate is increased. This family of curves, or preferably the computer model which solves these governing orbit equations, is preferably utilized to select the optimal RPM and chamber dimensions for the TVEMF-expansion of cells of a given sedimentation rate in a given external gravity field strength. Not to be bound by theory, but as a typical three-dimensional TVEMF culture is TVEMF expanded the tissues, cell aggregates, and tissue-like structures increase in size and sedimentation rate, and therefore, the rotation rate may preferably be adjusted to optimize the same.

In the three-dimensional TVEMF culture, the cell orbit (FIG. 6) from the rotating reference frame of the culture medium is seen to move in a nearly circular path under the influence of the rotating gravity vector (FIG. 8). Not to be bound by theory, but the two pseudo forces, coriolis and centrifugal, result from the rotating (accelerated) reference frame and cause distortion of the otherwise nearly circular path. Higher gravity levels and higher cell sedimentation rates produce larger radius circular paths which correspond to larger trajectory deviations from the ideal circular orbit as seen in the non-rotating reference frame. In the rotating reference frame it is thought, not to be bound by theory, that cells of differing sedimentation rates will remain spatially localized near each other for long periods of time with greatly reduced net cumulative separation than if the gravity vector were not rotated; the cells are sedimenting, but in a small circle (as observed in the rotating reference frame). Thus, in operation the present invention provides cells of differing sedimentation properties with sufficient time to interact mechanically and through soluble chemical signals. In operation, the present invention provides for sedimentation rates of preferably from about 0 cm/second up to 10 cm/second.

Furthermore, the present invention provides that, in operation, fresh or recycled culture medium may be moved within the rotatable perfusable culture chamber preferably at a rate sufficient to support metabolic gas exchange, nutrient delivery, and metabolic waste product removal. This may slightly degrade the otherwise quiescent three-dimensional TVEMF culture. In a preferred embodiment, cells which sediment at about 0.5 cm per second, and about 5 ml per minute culture medium perfused through a 500 ml rotatable perfusable culture chamber, an average flow-speed of about 0.001 cm per second is expected to result. Advantageously, this is far slower than either gravitationally or centrifugally induced cells motion. Preferably, the perfusion rate may be increased as the cells' metabolic demand increases and a large margin is available before significant fluid shear stress results from the perfusion. It is preferable, therefore, to introduce a mechanism for the support of preferred components including, but not limited to, respiratory gas exchange, nutrient delivery, growth factor delivery to the culture medium of the three-dimensional TVEMF culture, and also a mechanism for metabolic waste product removal in order to provide a long term three-dimensional TVEMF culture able to support significant metabolic loads for periods of hours to months.

In operation, the present invention not only provides for high concentrations of cells that maintain their three-dimensional geometry and cell-to-cell support but in addition, supplies TVEMF to the cells in the three-dimensional TVEMF culture that may affect some properties of cells during TVEMF-expansion. Without being bound by the theory, for instance up-regulation of genes promoting growth, or down regulation of genes preventing growth. In use, the present invention provides that the cells in the three-dimensional TVEMF culture are exposed to a TVEMF preferably of from about 0.05 gauss to about 6 gauss during TVEMF-expansion, more preferably of from about 0.05 gauss to about 0.5 gauss, and most preferably about 0.5 gauss. The electromagnetic field is generated by a TVEMF-source. In operation, the TVEMF-source of a rotatable perfused TVEMF-bioreactor may preferably be rotatable with the rotatable perfusable culture chamber, meaning about the same axis as the rotatable perfusable culture chamber preferably in the same direction. On the other hand, preferably the rotatable perfusable culture chamber may be rotated in the opposite direction of the TVEMF-source. Also, the TVEMF-source may preferably be fixed in relation to a rotatable perfusable culture chamber of a rotatable perfused TVEMF-bioreactor. The TVEMF-source may preferably be integral with, meaning affixed to, the rotatable perfusable culture chamber of a rotatable perfused TVEMF-bioreactor or may preferably be adjacent to, preferably in close proximity to, more preferably removably touching, the rotatable perfusable culture chamber of a rotatable perfused TVEMF-bioreactor.

The time varying electromagnetic field is produced by a varying electrical potential, preferably in the form of a delta wave, more preferably a differential square wave, and most preferably a square wave (following a Fourier curve), preferably having a frequency of about 10 cycles per second. Preferably a current of about 0.1 mA to about 1000 mA, and more preferably about 10 mA, produces a TVEMF extending at least several centimeters from the conductive material. Typically, the range of frequency and oscillating electromagnetic field strength is a parameter, which may be selected for achieving the desired stimulation of particular cells, and for providing the appropriate amount of up/down regulation of genes, ultimately promoting TVEMF-expansion of cells.

In addition to the qualitatively unique cells that are produced by the operation of the present invention, not to be bound by theory, an increased efficiency with respect to utilization of the total rotatable perfusable culture chamber volume for cell and tissue culture may be obtained due to the substantially uniform homogeneous suspension achieved. Advantageously, therefore, the present invention, in operation, provides an increased number of cells or total tissue or tissue-like structure in the same rotatable perfused TVEMF-bioreactor with less human resources. Many cell types may be utilized in this process including, but not limited to, mammalian, reptile, fish, yeast, and bacteria. Fundamental cell and tissue biology research as well as clinical applications requiring accurate in vitro models of in vivo cell behavior are applications for which the present invention and method of using the same provides an enhancement because, as indicated above and throughout this application, TVEMF-expanded cells and tissue of the present invention have essentially the same three-dimensional geometry and cell-to-cell support and cell-to-cell geometry as naturally-occurring, non-TVEMF-expanded cells and tissue.

The method of the present invention provides these three criteria above in a manner heretofore not obtained and optimizes a three-dimensional TVEMF culture, and at the same time, provides the TVEMF-expansion such that a sufficient rate of expansion (increase in number per volume, diameter in reference to tissue, or concentration) is detected in a sufficient amount of time. The present invention is well adapted to carry out the objects and obtain the ends and advantages mentioned herein, as well as those inherent therein. Without departing from the scope of the invention, it is intended that all matter contained herein be interpreted as illustrative and not limiting.

### Operative Method

In operation, a rotatable perfused TVEMF-bioreactor preferably having a rotatable perfusable culture chamber of from 100 ml to 3 L, is preferably first connected to a culture medium flow loop including gas exchange membranes, a pump, and ports and is then sterilized, preferably with ethylene oxide gas, and washed with sterile phosphate buffered saline (PBS), watered, and aerated. The rotatable perfusable culture chamber is completely filled with the appropriate culture medium for the cells to be cultured, with room only for any intended additional volumes of culture medium, cells, and/or other preferred components of the culture medium of the intended three-dimensional TVEMF culture. Preferrably a controlled environment incubator completely surrounds the rotatable perfused TVEMF-bioreactor and is preferably set for about 5% CO₂ and about 21% oxygen, and the temperature is preferably of from about 26° C. to about 41° C., and more preferably about 37° C.±2° C.

In a preferred embodiment, a culture medium flow loop is set at a rate adequate to allow timely equilibration of the dissolved gases with the external controlled incubator environment. Initially, a stabilized culture environment is created in the culture medium. The rotation may preferably begin at about 10 revolutions per minute (RPM), the slowest rate which produces a microcarrier bead orbital trajectory in which the beads do not accumulate appreciably at the chamber walls either by gravitational induced settling or by rotationally induced centrifugation. All of the adjustable rotating parts in the rotatable perfused TVEMF-bioreactor are also set at the same rotation (preferably 10 RPM) in order to provide essentially no relative motion of the three-dimensional TVEMF culture with respect to the chamber internal surfaces. In this way, the rotatable perfused TVEMF-bioreactor produces the minimal fluid velocity gradients and fluid shear stresses in the three-dimensional TVEMF culture.

Cell attachment substrates are introduced either simultaneously or sequentially with cells or tissue into the rotatable perfusable culture chamber to give an appropriate density, preferably 5 mg of cell attachment substrate per ml of culture medium, and preferably the cell attachment substrate for the anchorage dependent cells are microcarrier beads. The cells are preferably injected into the stabilized culture environment, preferably over a short period of time, preferably 2 minutes, so as to minimize cell damage while passing through the delivery system. The cells may preferably be injected into a rotatable perfusable culture chamber with a syringe preferably into an injection port, for instance that illustrated in FIGS. 4 and 5. The culture medium is then rotated about a horizontal axis.

After injection of the cells is complete, the chamber outer wall is quickly returned to initial rotation, preferably in less than one (1) minute, preferably to match the angular rotational rate of the rest of the system, more preferably 10 RPM, and thereby return the shear stress to the minimal level obtainable for the cells. During the initial loading and attachment phase, the cells are allowed to equilibrate for a short period of time, preferably of from 2 hours to 4 hours, more preferably for a time sufficient for transient flows to dampen out.

In a preferred embodiment, the culture medium perfusion rate is set to zero during cell or tissue loading and initial attachment (cells attaching to the attachment substrate, preferably microcarrier beads) so as to retain the cells within the rotatable perfusable culture chamber as opposed to drawing them through the filter and culture medium flow loop where severe cell damage would occur. The absence of perfusion induced mixing, delivery of nutrients and other preferred components of the culture medium, waste product removal, and respiratory gas exchange during this period is well tolerated due to the small total amount of initial cellular metabolism and the brevity of this condition. Disruption of the three-dimensional TVEMF culture could result from buoyant gas interfering with the near solid body three-dimensional TVEMF culture. Preferably, therefore, all free gas bubbles are purged, preferably via a port, to promote minimal disruption to the three-dimensional TVEMF culture.

Preferably after the initial loading and attachment phase (preferably 2 to 4 hours) the culture medium flow loop is set at a low flow speed (preferably 4.5 ml per minute) that does not interfere with the initial three-dimensional assembly process. As the TVEMF-expansion of the three-dimensional TVEMF culture progresses the size and sedimentation rate of the assembled tissue increases, the system rotational rates may be increased (increasing in increments preferably of from about 1 to 2 RPM from about 10 to 30 RPM or more) in order to reduce the gravitationally induced orbital distortion (from the ideal circular streamlines) of the now increased diameter tissue pieces.

During TVEMF-expansion, the rotational speed of the three-dimensional TVEMF culture in the rotatable perfusable culture chamber may be assessed and adjusted so that the cell mixture remains substantially at or about the horizontal axis. Increasing the rotational speed is warranted to prevent wall impact, which is detrimental to further three-dimensional growth of delicate structure. For instance, an increase in the rotation is preferred if the cells, tissue, or tissue-like structures in the three-dimensional TVEMF culture fall excessively inward and downward on the downward side of the rotation cycle and excessively outward and insufficiently upward on the upward side of the rotation cycle. Optimally, the user is advised to preferably select a rotational rate that fosters minimal wall collision frequency and intensity so as to maintain the three-dimensional geometry and cell-to-cell support and cell-to-cell geometry of the cells, tissue, or tissue-like structures. The preferred speed of the present invention is of from about 5 to about 120 RPM, and more preferably from about 10 to about 30 RPM.

The three-dimensional TVEMF culture may preferably be visually assessed through the preferably transparent rotatable perfusable culture chamber and manually adjusted. The assessment and adjustment of the cell mixture may also be automated by a sensor (for instance, a laser), which monitors the location of the cell stem cells within a TVEMF-bioreactor. A sensor reading indicating too much cell movement will automatically cause a mechanism to adjust the rotational speed accordingly.

Furthermore, preferably after the initial loading and attachment phase (2 to 4 hours) the TVEMF-source is turned on and adjusted so that the TVEMF output generates the desired electromagnetic field in the three-dimensional TVEMF culture in the rotatable perfusable culture chamber, preferably in a range of from 0.05 gauss to 6 gauss, more preferably of from about 0.05 gauss to about 0.5 gauss, and most preferably 0.5 gauss. The TVEMF can also be applied to the three-dimensional TVEMF culture during the initial loading and attachment phase. It is preferable that TVEMF is supplied to the three-dimensional TVEMF culture for the length of the culture time until the culture is terminated. Not to be bound by theory, but TVEMF-expansion not only provides for high concentrations of cells that maintain their three-dimensional geometry and cell-to-cell support but TVEMF may affect some properties of cells during TVEMF-expansion, for instance up-regulation of genes promoting growth, or down regulation of genes preventing growth.

The size of the wire coil, or preferably the annular wire heater, of the TVEMF-source, and number of times it is wound, are such that when a TVEMF, preferably in the form of a square wave, preferably of from about 0.1 mA to about 1000 mA, more preferably of from about 1 mA to about 10 mA, for instance 10 mA, is supplied to the wire coil, a TVEMF, preferably of from about 0.05 gauss to about 6 gauss, and more preferably of from about 0.05 to about 0.5 gauss, and most preferably 0.5 gauss, is generated within the three-dimensional TVEMF culture in the rotatable perfusable culture chamber of the rotatable perfused TVEMF-bioreactor. Preferably, the square wave has a frequency of about 2 to about 25 cycles/second, more preferably about 5 to about 20 cycles/second, and for example about 10 cycles/second. However, these parameters are not meant to be limiting to the TVEMF of the present invention, as such may vary based on other aspects of this invention. TVEMF may be measured for instance by standard equipment such as an EN131 Cell Sensor Gauss Meter.

The rapid cell and tissue expansion and increasing total metabolic demand may necessitate additional intermittent addition of preferable components enriching the culture medium in the three-dimensional TVEMF culture including, but not limited to, nutrients, fresh culture medium, growth factors, hormones, and cytokines. This addition is preferably increased as necessary to maintain glucose and other nutrient levels. During the rapid cell and tissue expansion, spent culture medium comprising waste may preferably be removed. The three-dimensional TVEMF culture may be allowed to progress beyond the point at which it is possible to select excellent cells orbits; at a point when gravity has introduced constraints which somewhat degrade performance in terms of a low shear three-dimensional TVEMF culture.

In a preferred embodiment of the present invention, preferably every 15 minutes during the total culture period the inner filter assembly is stopped and started at 15-second intervals, preferably for 1 minute, at least in order to clear cells from the inner filter assembly surfaces. This prevents accumulation of substrates, cells, and debris on the filter. In addition, samples of the cells in the three-dimensional TVEMF culture may be collected as desired. If the samples are to be collected via a syringe, the chamber outer wall may be temporarily stopped to allow practical handling.

### Example I

### Three-Dimensional Rat Bone Cell Culture

### Preparation

A 100 ml rotatable perfused TVEMF-bioreactor, illustrated in the preferred embodiment of FIG. 4, was prepared by washing with detergent and germicidal disinfectant solution (Roccal II) at the recommended concentration for disinfection and cleaning followed by copious rinsing and soaking with high quality deionized water. The rotatable perfused TVEMF-bioreactor was sterilized by autoclaving then rinsed once with culture medium.

### Inoculation

The rotatable perfused TVEMF-bioreactor was filled with culture medium consisting of minimum essential medium (MEM) with Earle's salts, growth supplements, antibiotics and 10% fetal bovine serum. After equilibration for one (1) hour in a CO₂ incubator, at 5% CO₂ environment at 37° C., the substrate consisting of collagen coated dextian polymer, Cytodex 3 microcarrier beads (Pharmacia Fine Chemicals, Uppsala, Sweden) were suspended in a small volume of culture medium and loaded into the rotatable perfusable culture chamber of the rotatable perfused TVEMF-bioreactor. An empty syringe attached to one of the sampling ports functioned as a compliant volume reservoir during inoculation to receive the displaced media. The final bead concentration was 5 mg/ml of chamber volume.

A volume of culture medium was injected to completely fill the chamber. All air bubbles were removed using a syringe attached to a sample port on the chamber. The rotatable perfusable culture chamber of the rotatable perfused TVEMF-bioreactor containing suspended beads was equilibrated for about 30 minutes.

Mono-dispersed primary rat osteoblast calvarium bone cells were suspended in a small volume of the same culture medium already in the rotatable perfusable culture chamber at a concentration of 5×10⁵ cells/ml. 50 ml at a concentration of 5×10⁵ cells/ml were then inoculated into the rotatable perfusable culture chamber by injection through a sample port. The cell seeding density was approximately 10 cells/bead. A second syringe was attached to another port and served as a compliant volume reservoir for the chamber. The motor was switched on and the rotatable perfused TVEMF bioreactor was rotated at a rate of approximately 16 RPM. The TVEMF source was also turned on to a gauss level of 0.05 gauss in a square wave form (following a Fourier curve).

### Cell Attachment and Three Dimensional Growth

At 24 hours, the rotation of the rotatable perfused TVEMF-bioreactor was stopped, a sample was collected through a syringe, and the rotation rate was again increased to the rate that was applied before the collection, approximately 16 RPM. The sample was analyzed under a microscope (400× magnification). Microscopic observation showed that the cells were well attached and flattened on the surface of the beads. The beads and cells were not associated into higher order structures at this point. Very little orbital path distortion or centrifugation was observed. The medium was changed by simultaneously removing medium with a syringe and adding a volume of the same with another to remove non-viable floating cells. On day three, the medium was changed again, as above, to assure nutrient supply to the cells. At day four cells were in good condition. To replenish nutrients yet retain growth factors secreted by the cells, three-fourths of the medium was removed and the same volume of fresh medium was added by using two syringes simultaneously as above. The same procedure was repeated on day 5.

On day 5, a sample of the three-dimensional TVEMF culture was again collected and microscopic observation of samples showed rounded assemblies of beads that were larger than seen on the previous day. Also noted were cells spanning spaces between beads. The airflow into the device was turned up to provide more oxygen to the rapidly growing culture. At this point the assemblies were observed to fall radially inwards on the “down” side and outwards on the “upside”.

On the 6th day the culture was transferred to a larger 250 ml volume rotatable perfused TVEMF-bioreactor, as in FIG. 5. On day 8, ordered assemblies of beads were very large (1-2 mm) with 8 to 15 beads in the assemblies and more three-dimensional structure was noted. The medium was changed at this time. At this point some accumulation of the tissue assemblies was occurring at the outer chamber wall. This centrifugation effect was quite gentle.

The cells were maintained in the three dimensional structure for 17 days with additional medium changes on days 9, 10 and 12 and additions of glucose on days 11, 15 and 16. The results were visually assessed by both the naked eye, and also via a microscope (400× magnification). There was no evidence of mechanical damage and the size of the cell/bead assemblies did not exceed the ability of the device to suspend the cells. The assemblies were of 1 to 2 mm in size and consisted of cells of mixed morphology which may be the indicia of beginning differentiation. The run was terminated by choice of the investigators. Prior to the TVEMF-expansion, the sample had 5×10⁵ cells/ml. After the TVEMF-expansion, the sample had 4.2×10⁶ cells/ml.

### Example 2

### Formation of Artificial Tissue in Suspension

### Preparation

A 500 ml rotatable perfused TVEMF-bioreactor consisting of a 500 ml cell rotatable perfusable culture chamber, a hollow fiber oxygenator, a prototype diaphragm pump, an in-line pH sensor, sample ports and a peristaltic pump for infusion of fresh medium were assembled, sterilized by ethylene oxide (ETO) and aerated for two days. The chamber was then loaded with phosphate buffered saline (PBS) to rinse and remove residual ETO. During this step, a leak was discovered in the oxygenator and unit was replaced using sterile techniques. The system was then loaded with culture medium comprising minimum essential medium (MEM) with Earle's salts, growth supplements, antibiotics and 10% fetal bovine serum and placed in the CO₂ incubator at a 5% CO₂ environment and 37° C. After remaining sterile for at least two days, the rotatable perfusable culture chamber was loaded with cells and substrate as described below.

### Inoculation

Cytodex 3 microcarrier beads (Pharmacia Fine Chemicals, Uppsala, Sweden) were reconstituted according to standard laboratory procedures, suspended in Microcarrier Medium (MM), containing 20% fetal calf serum, 100 units of penicillin/ml and 100 ug of streptomycin/ml, loaded into a 50 ml syringe and injected into the chamber. The bead density in the chamber was 5 mg/ml of chamber volume.

The system was then loaded with Baby Hamster Kidney (BHK21) cells, at 56 passages. To achieve this, ampules of frozen stock BHK21 cells were thawed. The cells were suspended in 50 ml of Microcarrier (MM) at a concentration of 0.75×10⁶ cells/ml and all 50 ml were injected into the rotatable perfusable culture chamber through a 20 ml syringe (2.5 injections) The final cell seeding density was approximately 6 cells/bead. Cells were loaded into the chamber at 9:30 AM.

The system parameters were as follows. The constant volume diaphragm pump was timed to circulate the culture medium at 4.5 ml/min. The pump rate was turned up to 20 ml/min four days after addition of cells and beads in order to increase the oxygen delivery to the reactor chamber. The pump system delivered 0.7 ml every two seconds. The rotation rate of the chamber and the blade members were set at 15 to 20 RPM and remained there during the test. The filter assembly rotation rate was 25 to 30 RPM. This produced a very low turbulence environment resulting in a cell/bead suspension upon introduction of those cells. The TVEMF source was also turned on to a gauss level of 0.05 gauss in a square wave form (following a Fourier curve).

### Cell Attachment and Three-Dimensional Growth

In order to assess the rate of attachment of cells to the substrate beads, samples of the three-dimensional TVEMF culture, containing cell-bead assemblies, were removed via a syringe for cell counts and microscopic observation at 2, 4, and 6 hours after initially loading the chamber with cells. Many of the cells attached to the beads within two hours and flattened on the surface of the beads, which is an essential state preliminary to growth of the cells. Early in this experiment, microscopic observation showed that some of the mono-dispersed cells clumped in groups of 10 to 30 cells. At 4 hours, these clumps of cells had attached to beads and were flattened on the surfaces. No orbital distortion or centrifugation effects were visible at this point.

At 24 hours some beads were covered with cells but there were also many floating cells. At this time, fresh medium was perfusion into the chamber through a port or passageway. Four hours later, a sample was collected and upon visual microscopic observation it appeared that almost all of the cells had attached to beads and there was no evidence of cell-cell aggregates as seen earlier. The poor appearance at 24 hours was probably due to toxicity from the new oxygenator used to replace the original one that leaked during the set up.

At 48 hours, the cells were visually assessed through the rotatable perfusable culture chamber and it was determined that the cell growth rate had increased rapidly. Glucose, glutamine (nutrients) and sodium hydroxide for pH control were added on day four to compensate for cell metabolism and depletion of nutrients.

On day 5 aggregates of cells and beads were visually noted and the medium was changed to minimum essential medium (MEM) with Earle's salts, growth supplements, antibiotics and containing 2% fetal calf serum, to slow cell metabolism and growth. The cells continued to grow, however, and reached a maximum density of 148 cells/bead at day 7. Very large and uniformed assemblies of about 1 to 2 mm formed and did not disaggregate during the remainder of the test. Ordered uniform assemblies of 8 to 10 beads formed in three-dimensional arrays over which the cells grew into a smooth membrane-like configuration of elongated fibroblastoid morphology. Multiple layers of these cells were apparent by microscope examination both on the surface and in the inter-bead spaces. The cells were placed in ordered layers with cell membranes immediately adjacent to each other. The test was terminated on day 10. The size of most of the tissue-like aggregates of cells on beads did not exceed the limits of the rotatable perfusable culture chamber of the rotatable perfused TVEMF-bioreactor to freely suspend the aggregates in a quiescent three-dimensional TVEMF culture, unrestricted by internal boundaries. Some very large assemblies, 3 to 8 mm, were observed. These rapidly sedimenting tissues were observed to exhibit grossly distorted orbital paths and centrifugation to the outer chamber walls. High-energy impacts and vigorous “rolling” effects were observed. These were considered beyond the process capacity to retain the quiescent, low shear, three-dimensional TVEMF culture. Prior to the TVEMF-expansion, the sample had 0.75×10⁶ cells/ml. After the TVEMF-expansion, the sample had 5.4×10⁶ cells/ml.

1. A rotatable perfused time varying electromagnetic force bioreactor comprising:
a rotatable perfusable culture chamber; and
a time varying electromagnetic force source comprising a coil, operatively connected to the rotatable perfusable culture chamber.

2. The rotatable perfused time varying electromagnetic force bioreactor as in claim 1, wherein the time varying electromagnetic force source is integral with the rotatable perfused time varying electromagnetic force bioreactor.

3. The rotatable perfused time varying electromagnetic force bioreactor as in claim 1, wherein the time varying electromagnetic force source is adjacent to the rotatable perfused time varying electromagnetic force bioreactor.

4. A method for producing time varying electromagnetic force-expanded cells in a rotatable perfused time varying electromagnetic force bioreactor, having a rotatable perfusable culture chamber and a time varying electromagnetic force-source comprising a coil, comprising the steps of:
a. filling the rotatable perfusable culture chamber with a culture medium;
b. placing cells in the rotatable perfusable culture chamber and initiating a three-dimensional time varying electromagnetic force culture;
c. rotating the rotatable perfusable culture chamber about an axis at a rotation speed;
d. controlling the rotation of the rotatable perfusable culture chamber by adjusting the rotation speed while maintaining the three-dimensional time varying electromagnetic force culture; and
e. exposing the cells to a time varying electromagnetic force to expand the cells.

5. The method of claim 4, wherein the time varying electromagnetic force is in the form of a square wave.

6. The method of claim 5, wherein the time varying electromagnetic force is of from about 0.05 gauss to about 6 gauss.

7. The method of claim 5, wherein the time varying electromagnetic force is of from about 0.05 gauss to about 0.5 gauss.

8. The method of claim 5, wherein the square wave has a frequency of from about 2 to about 25 cycles/second.

9. The method of claim 5, wherein the square wave has a frequency of from about 5 to about 20 cycles/second.

10. The method of claim 5, wherein the square wave has a frequency of 10 cycles/second.

11. The method of claim 4, wherein the three-dimensional time varying electromagnetic force culture has the properties of collocation of the culture medium and the cells, essentially no relative motion of the culture medium with respect to the rotatable perfusable culture chamber, and freedom for a three-dimensional spatial orientation of the cells.

12. The method of claim 4, wherein the three-dimensional time varying electromagnetic force culture is maintained by perfusion.

13. The method of claim 4, wherein the culture medium is enriched by a culture medium flow loop comprising a supply manifold, a pump, an oxygenator, a rotatable perfusable culture chamber, and a waste.

14. The method of claim 4, wherein prior to step c, the culture medium flow loop is turned on.

15. The method of claim 13, wherein the culture medium flow loop enriches the culture medium with at least one selected from the group consisting of growth factors, cytokines, hormones, oxygen, nutrients, acids, bases, buffers, and fresh culture medium prior to entering the rotatable perfusable culture chamber.

16. The method of claim 4 wherein the rotatable perfused time varying electromagnetic force-bioreactor is located in unit gravity.

17. The method of claim 4 wherein the rotatable perfused time varying electromagnetic force-bioreactor is located in microgravity.

18. The method of claim 4, wherein the rotatable perfused time varying electromagnetic force-bioreactor is located in less than unit gravity.

19. The method of claim 4, wherein the culture medium further comprises at least one cell attachment substrate.

20. The method of claim 4 wherein the rotation speed is of from about 5 to about 120 RPM.

21. The method of claim 4 wherein the rotation speed is 10 RPM.

22. The method of claim 4, wherein the cells maintain essentially the same three-dimensional geometry, and cell-to-cell support and geometry as that of the cells found in vivo.

23. The method of claim 4, wherein the time varying electromagnetic force is in the form of a differentiated square wave.

24. The method of claim 4, wherein the time varying electromagnetic force is in the form of a delta wave.

25. The method of claim 4, wherein the cells are expanded to more than seven times their original number.
